(19) United States
(12) Patent Application Publication
(10) Pub. No.: US 2023/0292720 A1
(43) Pub. Date: Sep. 21, 2023

(54) PRECISE BREEDING METHODS FOR GENE-EDITED NON-HUMAN ANIMALS

(71) Applicant: Agricultural Technology Research Institute, Hsinchu City (TW)

(72) Inventors: Ching-Fu Tu, Hsinchu City (TW); Chin-Kai Chuang, Hsinchu City (TW); Shu-Hui Peng, Hsinchu City (TW); Yu-Hsiu Su, Hsinchu City (TW)

(73) Assignee: Agricultural Technology Research Institute, Hsinchu City (TW)

(21) Appl. No.: 18/184,017

(22) Filed: Mar. 15, 2023

Related U.S. Application Data
(60) Provisional application No. 63/319,920, filed on Mar. 15, 2022.

(51) Int. Cl. A01K 67/027; C12N 9/22; C12N 5/077

(52) U.S. Cl. CPC A01K 67/0276; C12N 9/22; C12N 5/0656; C12N 2310/20; A01K 2227/108

(57) ABSTRACT

The present invention relates to a breeding method for generating gene-edited non-human animals. In particular, the method of the present invention features simultaneous reprograming and gene-editing carried out in somatic cells and subsequent subcloning and genotyping conducted at the in vitro cell stage to obtain precisely gene-edited induced pluripotency stem cells (iPSCs) which are then used in somatic nuclear transfer (SCNT) to generate a precisely gene-edited non-human animal embryo and a resultant gene-edited non-human animal.

### RELATED APPLICATIONS

This application claims the benefit of U.S. provisional application No. 63/319,920, filed Mar. 15, 2022 under 35 U.S.C. § 119, the entire content of which is incorporated herein by reference.

Reference to an Electronic Sequence Listing

The contents of the electronic sequence listing (ATI0032US.xml; Size: 24,889 bytes; and Dated of Creation: Mar. 9, 2023) is herein incorporated by reference in its entirety.

### TECHNOLOGY FIELD

The present invention relates to a breeding method for generating gene-edited non-human animals. In particular, the method of the present invention features simultaneous reprograming and gene-editing carried out in somatic cells and subsequent subcloning and genotyping conducted at the in vitro cell stage to obtain precisely gene-edited induced pluripotency stem cells (iPSCs) which are then used in somatic nuclear transfer (SCNT) to generate a precisely gene-edited non-human animal embryo and a resultant gene-edited non-human animal.

### BACKGROUND OF THE INVENTION

Takahashi and Yamanaka [1] reported only four transcription factors, including Oct4, Sox2, Klf4 and c-Myc (OSKM), are needed for inducing mouse induced pluripotency stem cells (iPSCs). The same group [2] and Yu et al. [3] used OSKM and Oct4/Sox2/Lin28/Nanog transcription factors respectively to generate human iPSC. Some research teams reported generation of porcine iPSC [4-6]. West et al. [7, 8] reported germline transmitted chimeric pigs produced from porcine iPSC. Liu et al. generated porcine iPSC using porcine Oct4 and Klf4 together with small molecules and confirmed its pluripotency by formation of teratoma [9]. Some teams described morula injected with iPSC or cloned 4-cell embryos aggregation [10]. Further, cloned early stage embryos [10-13] and live piglets [14, 15] were generated using porcine iPSC as nuclear donors for somatic cell nuclear transfer (SCNT).

Gene editing (GE) technologies have been developed in this art, including those based on zinc finger nuclease (ZFN) [17], transcription activator-like effector nuclease (TALEN) [18], and CRISPR/Cas9 (clustered regularly interspaced short palindromic repeats and the associated protein 9) [19]. The CRISPR/Cas9 technology is regarded as a canonical methodology with high efficiency and rapid realization, a low technological barrier and low cost that can be broadly applied in many organisms. Serval modified forms of Cas9 are available which can more precisely create a single strand of target DNA nicked or single base edited [20]. Up to date, most of the iPSC applications are concentrated on human cellular therapeutics especially for cancer or genetic diseases. Howden et al. [21] was first one used patients' fibroblasts, one adult with retinitis pigmentosa and the other infant with severe combined immunodeficiency, for simultaneously reprogramming and gene correcting to generate the DNMT3B and Oct4 with green fluorescent protein (GFP) genes KI iPSC, while their iPSC subclones had only 3 to 5% expression GFP. Wang et al. [22] used skin fibroblasts from psychotic disorder patients and generated CHD8 hemizygosity iPSC to study the autism spectrum disorders. Kim et al. [23] used fibrodysplasia ossificans progressiva (FOP) syndrome patient's fibroblasts to generate active bone morphogenetic protein type I receptor (also called ALK2) gene-corrected (ALK2 c.617G4A) iPSCs. Bell et a. [24] established a rapid pipeline to develop a targeted therapeutic model for rare neurodevelopmental disorders. Tidball et al. [25] rapidly generated human genetic loss-of-function iPSC lines. Melo et al. [26] and Wen et al. [27] used erythroblast, which easily collected cells source from a healthy 97-year-old woman, and peripheral blood mononuclear cells (PBMC), respectively, to generate CAPN1 mutated iPSC and to establish high-level precise knockin iPSC, which genome editing was performed with a bulk iPSC population without any selection. Howden et al. [28] developed a model for human ‘brittle bone’ disease, osteogenesis imperfecta (00, and established and corrected the patient COL1A1 c.3936G>T iPSC line. Ye et al. [29] established Alzheimer's (Alz) disease patient-derived iPSC carrying three copy of APP (amyloid precursor protein) gene to study the dysregulation of gene dosage in Alz disease. However, no reports describe applications of iPSC with gene edition in breeding of non-human animals.

Since late 1980s, porcine reproductive and respiratory syndrome virus (PRRSV) rapidly emerged and became an epidemic devastating the pig industry globally. In vivo, the virus shows very narrow cell tropism, targeting specific subsets of porcine monocytes/macrophages, and it infects the cells via the heparan sulfate, sialoadhesin (CD169) and CD163 receptors [30]. To date, CD163 on porcine macrophages has been the best-studied receptor involved in PRRSV infection [31]. Efforts including the KO of CD163 [32-35], deletion of exon 7 (scavenger receptor cysteine-rich domain 5 (SRCR5) region of the CD163 protein) of the CD163 gene [36, 37], and the deletion of a portion of exon 7 in the infective pocket of virons [38] have achieved full resistance to PRRSV infection without disturbing the well-being of GE pigs [33, 37-41]. However, fundamental limitation to the precision of gene edition for breeding non-human animals having desired gene edition and traits still remain

Therefore, there is a need to develop more precise and efficient breeding techniques for generating gene-edited non-human animals, especially large domestic animals.

### SUMMARY OF THE INVENTION

The present invention provides a precise breeding method for gene-edited non-human animals. In particular, the method of the present invention features simultaneous reprograming and gene-editing carried out in somatic cells and subsequent subcloning and genotyping conducted at in vitro cell stage to obtain precisely gene-edited iPSCs which are then used in somatic nuclear transfer (SCNT) to generate a precisely gene-edited non-human embryo and a resultant gene-edited non-human animal.

In one aspect, the present invention provides a method of producing a genetically edited non-human embryo and a resultant genetically edited non-human animal, comprising
(a) gene editing non-human mammalian somatic cells to induce a gene edition of interest and simultaneously reprograming the cells to reprogram into induced pluripotency stem cells (iPSCs), so as to produce a plurality of gene-edited iPSC candidates;
(b) subcloning and genotyping the gene-edited iPSC candidates to obtain a gene-edited iPSC subclone having a genome with the gene edition of interest;
(c) transferring the gene-edited iPSC subclone into an enucleated oocyte to generate a reconstituted embryo; and
(d) culturing the reconstituted embryo to reach the blastocyst stage to give rise to a non-human gene-edited animal with the gene edition of interest.

In some embodiments, the gene editing is CRISPR/Cas9-based gene editing.

In some embodiments, the reprogramming factors comprises Klf4, c-Myc, Nanog, Oct4, Sox2 and SV40 large T antigen.

In some embodiments, the somatic cells are simultaneously transfected by CRISPR/Cas9-based gene editing vectors and reprogramming vectors.

In some embodiments, the CRISPR/Cas9-based gene editing vectors comprise
a Cas9 vector comprising nucleic acids encoding a Cas9 protein, and
one or more gRNA vectors each comprising nucleic acids encoding a gRNA molecule for targeting Cas9 to a gene of interest to induce the gene edition.

In some embodiments, the reprogramming vectors comprise
a first reprogramming vector comprising nucleic acids encoding Oct4, Sox2, Klf4 and Nanog;
a second reprogramming vector comprising nucleic acids encoding c-Myc; and
a third reprogramming vector comprising nucleic acids encoding SV40 large T antigen.

In some embodiments, the somatic cells and the oocyte are from the same species.

In some embodiments, the somatic cells are fibroblasts.

In some embodiments, the gene edition is gene knock-in or gene knock-out or partial deletion.

In some embodiments, the non-human animal is selected from the group consisting of sheep, cattle, deer, goat, monkeys, camels and pigs.

In some embodiments, the gene edition is gene knock-out or partial deletion of CD163.

Specifically, the present invention provides a method of providing a CD163 gene-edited pig, comprising
(a) simultaneously transfecting porcine somatic cells with gene editing vectors and reprogramming vectors, wherein the gene editing vectors provide gene edition of a CD163 gene, so as to produce a plurality of CD163 gene-edited porcine iPSC (piPSC) candidates;
(b) subcloning and genotyping the gene-edited piPSC candidates to obtain a CD163 gene-edited piPSC subclone having a genome with the gene edition of the CD163 gene;
(c) transferring the CD163 gene-edited iPSC subclone into an enucleated porcine oocyte to generate a reconstituted porcine embryo; and
(d) culturing the reconstituted porcine embryo to reach the blastocyst stage to give rise to a pig with the gene edition of the CD163 gene.

In particular, the resultant pig with the gene edition of the CD163 gene exhibits resistance to porcine reproductive respiratory syndrome virus (PRSV) infection.

Also provided is a method to provide resistance to porcine reproductive respiratory syndrome virus (PRSV) infection in a pig, comprising
(a) simultaneously transfecting porcine somatic cells with gene editing vectors and reprogramming vectors, wherein the gene editing vectors provide gene knockout or partial deletion of a CD163 gene, so as to produce a plurality of CD163 gene-edited porcine iPSC (piPSC) candidates;
(b) subcloning and genotyping the gene-edited piPSC candidates to obtain a population of CD163 gene-edited piPSC subclones having a genome with gene knockout or partial deletion of the CD163 gene;
(c) transferring each of the CD163 gene-edited iPSC subclones into an enucleated porcine oocyte to generate reconstituted porcine embryos;
(d) culturing the reconstituted porcine embryos to reach the blastocyst stage to give rise to a plurality of CD163 gene-edited pigs; and
(e) selecting a pig line from the plurality of CD163 gene-edited pigs generated in (d) that exhibits resistance to PRSV as compared with a non gene-edited pig counterpart growing under the same conditions

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

The patent or application file contains at least one drawing executed in color. Copies of this patent or patent application publication with color drawing(s) will be provided by the Office upon request and payment of the necessary fee.

In the drawings:

FIG. 1A-1D shows schematic diagrams of the recombinant Cas9 protein and single-guide RNA expression vectors. FIG. 1A shows the linear map of the pCX-Flagg-NLS₁-Cas9-NLS₂ plasmid DNA vector and NLS₁-Cas9-NLS₂ recombinant protein. FIG. 1B shows the linear map of the ppU6-(BsaI)2-gRNA plasmid DNA vector, and the DNA sequence (SEQ ID NO:1) covering the pU6 promoter, the BsaI cutting sites and the guide RNA coding region. FIG. 1C shows a sketch to present how to build a single-guide RNA (sgRNA) expression vector by a simply ligation reaction.

FIG. 1D shows the sequences of two proto-spacers including proto-spacer 26 (forward: SEQ ID NO: 8, reverse: SEQ ID NO: 9) and proto-spacer 28 (forward: SEQ ID NO: 12, reverse: SEQ ID NO: 13), and two primer pairs for constructing sgRNA expression vector for the spacers including primer pCD163-Sp26F (SEQ ID NO: 6), pCD163-Sp26R (SEQ ID NO: 7), pCD163-Sp28F (SEQ ID NO: 10) and pCD163-Sp28R (SEQ ID NO: 11) for cloning of CD163 single-guide RNA vectors.

FIG. 2A-2B shows the sites of gene editing to precisely remove the exon 7 of pig CD163 gene. FIG. 2A shows the schematic map of pig CD163 gene, from exon 6 to exon 8 including proto-spacer 26 (Sp26) and proto-spacer 28 (Sp28). FIG. 2B shows porcine genomic DNA sequences near CD163 exon 7 (SEQ ID NO:14). The Cas9 cutting sites are indicated as “sgSL26 cut” and “sgSL28 cut”.

FIG. 3A-3C shows the generation of pig CD163 exon? edited/deleted (CD163ΔE7) porcine induced pluripotent stem cell (piPSC) by transfection iPSC induced factors and CRISPR/Cas9 gene-editing plasmid vectors by electroporation. FIG. 3A shows the morphology of piPSC after twice (2^{nd} EP), thrice (3^{rd} EP), or fourth (4^{th} EP) electroporation; the treated primary fibroblast cells were established from pigs, L259-10 and D529-16. FIG. 3B shows the primer-pair, F2 (SEQ ID NO: 15) and R2 (SEQ ID NO: 16), which are used in PCR amplification to amplify a DNA fragment involving the two proto-spacers to check gene deletion efficacy. The sequences on porcine DNA to be recognized by the primers F2 and R2 are as indicated in FIG. 2A. Candidate piPSC were analyzed by genomic DNA PCR, the red color are homologous piPSCs carrying double chromosome CD163ΔE7 and the green colors are heterologous; in parentheses, the numbers indicate No. of candidate/No. of analyzed and % mean efficiency of homologous CD163ΔE7. FIG. 3C shows that the PCR amplicon from the homologous CD163ΔE7 piPSCs were further analyzed by PCR amplicon sequencing and confirmed being CD163ΔE7. The clones have SEQ ID NO: 17 lacking exon 7 from 23268 bp to 23753 bp showing PCR amplicon in length of 454 bp.

FIG. 4 shows the cloned porcine blastocysts by using CD163ΔE7 piPSC. The maturated IVM oocytes were enucleated and the CD163ΔE7 piPSC were directly microinjected into their cytoplasm. After 6 to 7 days cultivated in PZMS medium, the reconstituted porcine blastocyst could be obtained (shown by the arrows).

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of”

As used herein, the term “nucleic acid” or “polynucleotide” can refer to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

As used herein, the term “polypeptide” refers to a polymer composed of amino acid residues linked via peptide bonds. The term “protein” typically refers to relatively large polypeptides. The term “peptide” typically refers to relatively short polypeptides (e.g., containing up to 100, 90, 70, 50, 30, 20 or 10 amino acid residues).

As used herein, the term “encoding” refers to the natural property of specific sequences of nucleotides in a polynucleotide (e.g., a gene, a cDNA, or an mRNA) to serve as templates for synthesis of other polymers and macromolecules in biological processes having either a given sequence of RNA transcripts (i.e., rRNA, tRNA and mRNA) or a given sequence of amino acids and the biological properties resulting therefrom. Therefore, a gene encodes a protein if transcription and translation of mRNA produced by that gene produces the protein in a cell or other biological system. As used herein, a “coding sequence” or a sequence “encoding” an expression product, such as an RNA or polypeptide, is a nucleotide sequence that, when expressed, results in the production of that RNA or polypeptide i.e., the nucleotide sequence encodes an amino acid sequence for that polypeptide. A coding sequence for a protein may include a start codon (usually ATG) and a stop codon. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” encompasses all nucleotide sequences that are degenerate versions of each other and that encode the same amino acid sequence.

As used herein, the term “gene” refers to a nucleic acid comprising an open reading frame encoding a polypeptide of interest, including both exon and (optionally) intron sequences. A gene can be a DNA sequence that is transcribed into RNA and in some instances translated into a polypeptide in vivo when placed under the control of appropriate regulatory sequences. For example, a gene of interest may encode an RNA transcript, a native polypeptide (i.e. a polypeptide found in nature) or fragment thereof, an engineered polypeptide or peptide fragment and the like.

The terms “gene editing” or “genome editing” as used interchangeably herein, is a type of genetic engineering in which DNA is deleted, inserted and/or replaced in the genome of a targeted cell. Targeted gene editing (interchangeable with targeted genomic editing) may include deletion, insertion and/or substitution at one or more given sites in the genome. When an endogenous sequence is deleted during the gene editing, an endogenous gene comprising the affected sequence may be knocked-out or knocked-down as a result of the sequence deletion. Therefore, targeted gene editing may be used to disrupt endogenous gene expression. Gene editing can be achieved through specific introduction of double stand breaks by specific endonucleases. Examples of genome editing include zinc finger, TALEN, and CRISPR/Cas9, which are known and available in this art, wherein CRISPR is preferable.

CRISPR/Caspase-9 requires two major components: (1) a Caspase-9 endonuclease (Cas9) and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction.

As used herein, the term “recombinant polynucleotide” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence or maintaining the given nucleotide sequence for replicating it, manipulating it or transferring it between different locations (e.g., between different organisms). Vectors can be introduced into a suitable host cell for the above-mentioned purposes. The term “operably linked” may mean that a polynucleotide is linked to an expression control sequence in such a manner to enable expression of the polynucleotide when a proper molecule (such as a transcriptional factor) is bound to the expression control sequence. The term “expression control sequence” or “regulatory sequence” means a DNA sequence that regulates the expression of the operably linked nucleic acid sequence in a host cell. Examples of vectors include, but are not limited to, plasmids, cosmids, phages, YACs or PACs. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprise, for example and without limitation, a promoter sequence (e.g., a cytomegalovirus (CMV) promoter, simian virus 40 (SV40) early promoter, a lac promoter, a T7 promoter, and alcohol oxidase gene (AOX1) promoter), a start codon, a replication origin, enhancers, an operator sequence, a secretion signal sequence (e.g., α-mating factor signal) and other control sequence (e.g., Shine-Dalgano sequences and termination sequences). Preferably, vectors may further contain a marker sequence (e.g., an antibiotic resistant marker sequence) for the subsequent screening procedure.

As used herein, the term “totipotent” or “totipotency” refers to the ability of a cell to divide and ultimately produce an entire organism including extra embryonic tissues. For example, a fertilized oocyte is a totipotent stem cell which gives rise to all of the embryonic and extraembryonic tissues of an organism. Specifically, the term “totipotent” may indicate the cell's ability to progress through a series of divisions into a blastocyst. The blastocyst comprises an inner cell mass (ICM) and an outer-layer of cells, called trophoblast. Trophoblast cells eventually form extra-embryonic tissues, including placenta and amnion. The cells found in the ICM result in pluripotent stem cells which have the ability to proliferate indefinitely, and when properly induced, differentiate in different cell types in the body.

As used herein, the term “pluripotent” or “pluripotency” means the ability of a cell to self-renew and to differentiate into all cell lineages. For example, embryonic stem cells (ESCs) are a type of pluripotent stem cells (PSCs) which can form cells from each of the three germs layers, the ectoderm (interior stomach lining, gastrointestinal tract, the lungs), the mesoderm (muscle, bone, blood, urogenital), and the endoderm (epidermal tissues and nervous system). PSCs can give rise to various fetal or adult cell types including germ cells. However, PSCs alone cannot produce a fetal or adult animal when transplanted into utero since they do not have the potential to contribute to extra embryonic tissues e.g. placenta.

As used herein, the term “differentiation” means a process of cell development where the cell differentiates to acquire specific features to make it perform a specific function. Differentiation is a relative process. Mature somatic cells e.g. osteoblasts (bone), chondrocytes (cartilage), adipocytes (fat), fibroblasts (skin), hepatocytes (liver) can be terminally differentiated that already lose the ability to differentiate into different cell types.

As used herein, the term “reprogramming” or “dedifferentiation” refers to a process of increasing the potency of a cell or dedifferentiating the cell to a less differentiated state. Specifically, a reprogrammed cell is one that is in a less differentiated state than the same cell in a non-reprogrammed state. Reprogramming can be induced by using reprogramming factors or chemicals.

As used herein, the term “induced pluripotent stem cells” or iPSCs are pluripotent stem cells which are reprogrammed from adult tissues or differentiated cells (e.g. somatic cells). iPSCs are considered having the same pluripotent characteristics as the natural pluripotent stem cells (e.g. ESCs) while iPSCs do not refer to cells as they are found in nature. iPSCs can be produced by insertion of one or more specific genes or stimulation with chemicals in somatic cells.

As used herein, the term “zygote” refers to a cell formed when two gamete cells are jointed. It is the earliest development stage of an embryo. Zygotes are produced by fertilization between two haploid cells, an ovum (female gamete) and a sperm cell (male gamete), which combine to form the single diploid cell.

As used herein, the term “embryo” refers to a cellular mass generated by one or more cell division of a zygote or an activated oocyte artificially incorporated with a source of cell nucleus.

As used herein, the term “morula” refers to a stage of embryonic development. The zygote, after about 3-4 days, forms a berry-like cluster of cells by a series of cleavages, called a morula, generally composed of 12-32 cells (called blastomeres). Through cellular differentiation and cavitation, the morula gives rise to the blastocyst. During blastocyst formation, the morula's cells differentiate into an inner cell mass growing inside the blastocoel and trophoblast cells forming the outer blastocoel membrane.

As used herein, the term “blastocyst” refers to a structure formed in the early development of mammals. A blastocyst forms at about 5-7 days after fertilization. It is generally a fluid-filled of about 60-100 cells sphere made up (i) an outer layer of cells (the trophectoderm), (ii) a cluster of cells on the interior (the inner cell mass, ICM, a source of embryonic stem cells), and (iii) a fluid-filled cavity (the blastocoel or blastocyst cavity).

As used herein, a “reconstructed embryo” is meant the cell which is formed by insertion of the donor cell or nucleus of the donor cell into the enucleated oocyte which corresponds to a zygote.

As used herein, the term “subcloning” refers to a progressive dilution of cells in series, for example, in a 96-well microliter plate, in order to obtain single colonies.

As used herein, the term “genotyping” refers to the determination of the genetic information a cell or an individual carries at one or more positions in the genome. For example, genotyping may comprise the determination of which allele or alleles an individual carries for genetic variants such as single nucleotide polymorphisms (SNPs) or insertion, deletion and/or substitution. For example, a specific nucleotide in a genome may be a T in some individuals and a G in other individuals. Those individuals who have a T at the polymorphic position have the T allele and those who have a G have the G allele. In a diploid organism, the individual will have two copies of the sequence containing the polymorphic position such that the individual may have a T allele and a G allele or alternatively two copies of the T allele or two copies of the G allele. Those individuals who have two copies of the T allele are homozygous for the T allele, those individuals who have two copies of the G allele are homozygous for the G allele, and those individuals who have one copy of each allele are heterozygous. In certain examples described herein, genotyping may comprise the determination of which allele or alleles a pig iPSC clone carries at the position of exon 7 between proto-spacer 26 and protospacer 28 (from 23268 bp to 23753 bp) in the genomic DNA of CD163. For example, those iPSC clones having a full-length fragment at exon 7 between proto-spacer 26 and protospacer 28 have the full-length exon 7 (F) allele and those who have deletion at exon 7 between proto-spacer 26 and protospacer 28 (from 23268 bp to 23753 bp) have the deletion exon 7 (D) allele. Those iPSC clones may have homogenous full-length exon 7 in both alleles, homogenous exon 7 deletion in both alleles, or heterogenous full-length exon 7 in one allele and exon 7 deletion in another allele.

According to the present invention, a method for generating gene-edited non-human animal embryo and a resultant gene-edited non-human animal is provided which features simultaneous reprograming and gene-editing carried out in somatic cells and subsequent subcloning and genotyping conducted at the in vitro cell stage to obtain gene-edited iPSC subclones which are then transferred to enucleated oocytes from which a gene-edited non-human animal embryo is generated that give rise to a gene-edited non-human animal.

Non-human animals of the present invention are typically non-human mammal animals. In some embodiments, a non-human mammal is a primate, a goat, a sheep, a pig, a dog, a cow, or a rodent.

Somatic cells of the invention may be primary cells (non-immortalized cells), such as those freshly isolated from an animal, or may be derived from a cell line (immortalized cells). Differentiated somatic cells are suitable starting cells in the methods. Examples of somatic cells include, but are not limited to fibroblasts, muscle cells, keratinocytes, and hepatocytes. They may be obtained by methods known in this art and can be obtained from suitable organs or tissue containing live somatic cells, e.g. skin.

In the present invention, somatic cells are reprogrammed to iPSCs. Reprograming can be accomplished by using a plurality of reprograming factors. The reprogramming factor may comprise one or more gene products which may be introduced into a cell by transducing the call with a recombinant vector comprising a gene encoding the reprogramming factor. Thus, the cell can express the reprogramming factor expressed as a product of a gene contained in the recombinant vector, thereby inducing reprogramming of a differentiated cell. In particular embodiments, the reprogramming factor comprises a plurality of gene products: an Oct family gene, a Klf family gene, a Sox family gene and a Myc family gene. Examples of Oct family gene include, for example, Oct3/4, Oct1A, Oct6, and the like. Examples of the Klf family gene include Klf1, Klf2, Klf4, Klf5 and the like. Examples of the Myc family gene include c-Myc, N-Myc, L-Myc and the like. Examples of the Sox family genes include, for example, Sox1, Sox2, Sox3, Sox7, Sox15, Sox17 and Sox18. In some embodiments, the reprogramming factor further comprises one or more of the following genes: Nanog, SV40 large T antigen, Fbx15, ERas, Tc11, Grb2, Gdf3, Rex1, ECAT1, ECAT8, ECAT15-1, ECAT15-2, Stella, Stat3 and Sa114. In particular examples, the reprogramming factor comprise Oct4, Sox2, Klf4, c-Myc, Nanog, and SV40 large T antigen. After reprogramming, iPSC candidates can be picked out by morphological characterization e.g. round shape, large nucleolus, and scant cytoplasm as similar to ESCs. In general, reprogrammed colonies are tightly packed, sharp edged and flat, and also mitotically active due to the possession of property of self-renewal. iPSC candidates may be further confirmed on the basis of expression of certain cell surface proteins, e.g., SSEA-4, alkaline phosphatase, and transcription factors, e.g., Oct4, Sox2, and Nanog. A variety of standard approaches may be used to determine, for example, for RNA levels, including, but not limited to, polymerase chain amplification, ribonuclease protection (RNase protection) assay, and Northern blot analysis, and for protein levels, including, but not limited to, enzyme-linked immunosorbent assay (ELISA), Western blot and intracellular staining.

In the present invention, gene editing is carried out simultaneously with reprogramming. Gene editing may involve genetic engineering in which nucleotide(s)/nucleic acid(s) is/are inserted, deleted, and/or substituted in a DNA sequence, such as in the genome of a targeted cell. Targeted gene editing enables insertion, deletion, and/or substitution at pre-selected sites in the genome of a targeted cell. When a sequence of an endogenous gene is edited, for example by deletion, insertion or substitution of nucleotide(s)/nucleic acid(s), the endogenous gene comprising the affected sequence may be knocked-out due to the sequence change. Therefore, targeted editing may be used to disrupt endogenous gene expression or function. A “disrupted gene” refers to a gene comprising an insertion, deletion or substitution relative to an endogenous gene such that expression of a functional protein from the endogenous gene is reduced or inhibited or the function of the protein is blocked or diminished. In some embodiments, a cell that comprises a disrupted gene does not express a detectable level of the protein encoded by the gene. In some embodiments, a cell that comprises a disrupted gene may express a truncated form of the protein encoded by the gene which is deficient in function. Examples of a gene to be edited include but are not limited to CD163, PDX1 and RAG2/IL2RG2.

Conventional gene editing methods can be used in the present invention. A nuclease-independent approach may include homologous recombination which is guided by homologous sequences flanking an exogenous polynucleotide to be introduced into an endogenous sequence through the enzymatic machinery of the host cell. A nuclease-dependent approach utilizes DNA repair mechanisms through the specific introduction of double strand breaks (DSBs) by specific rare-cutting nucleases (e.g., endonucleases). In some embodiments, gene disruption may occur by deletion of a genomic sequence using two guide RNAs in CRISPR/Cas9 gene editing technology. Methods of using CRISPR/Cas9 gene editing technology to create a genomic deletion in a cell are known in this art.

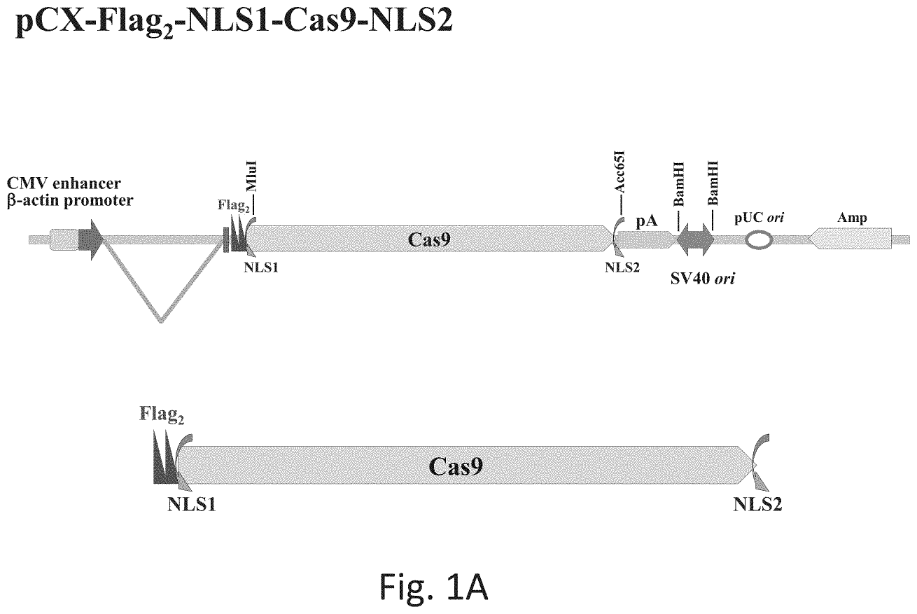

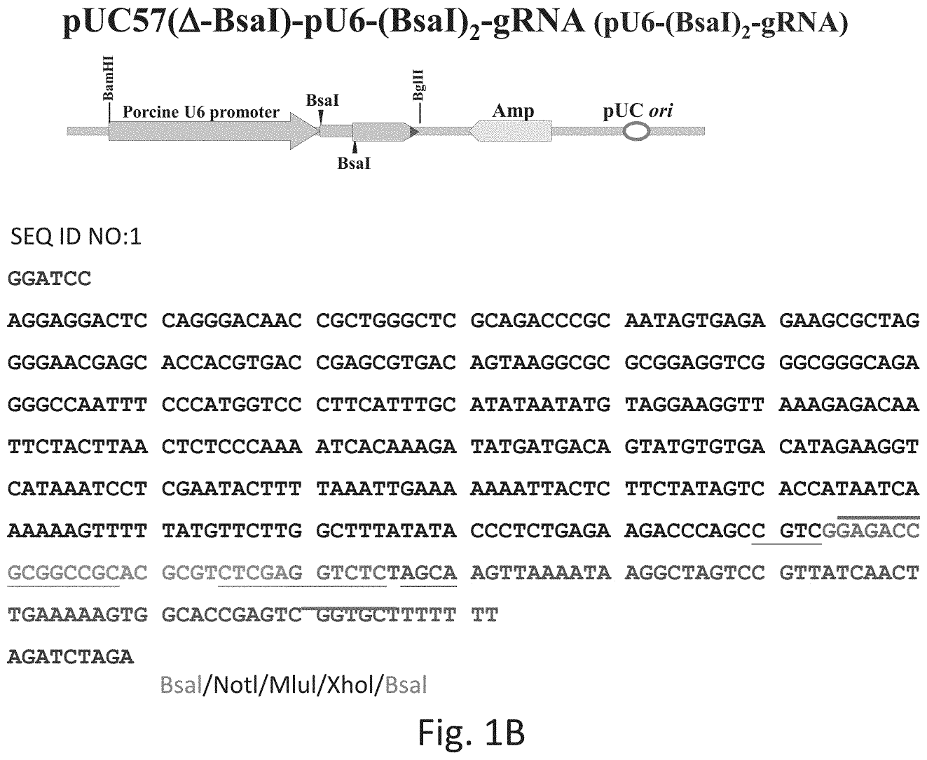

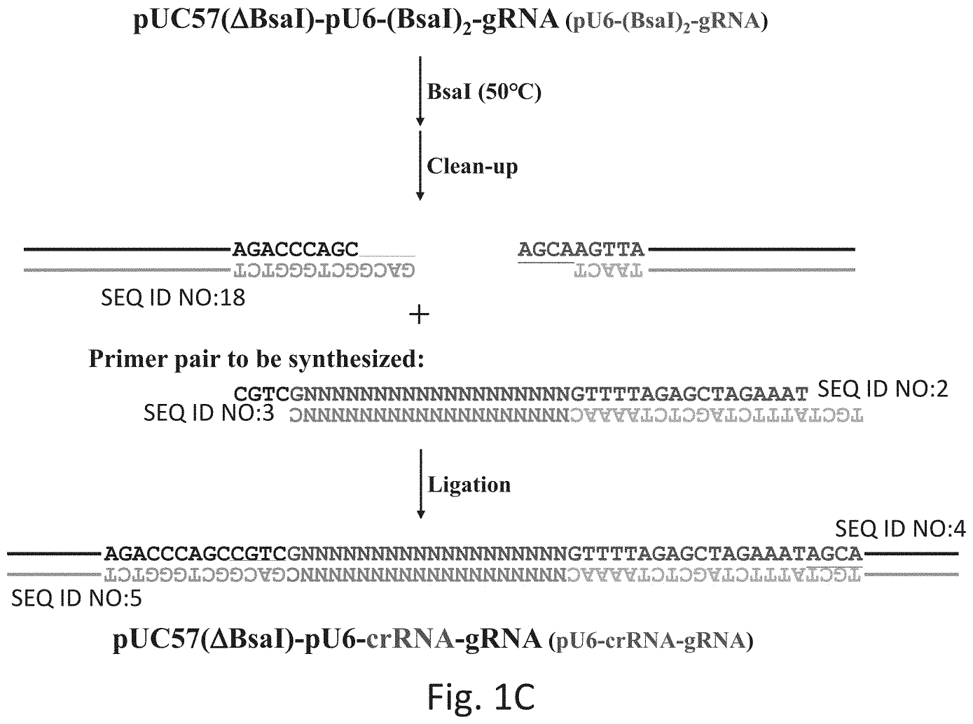

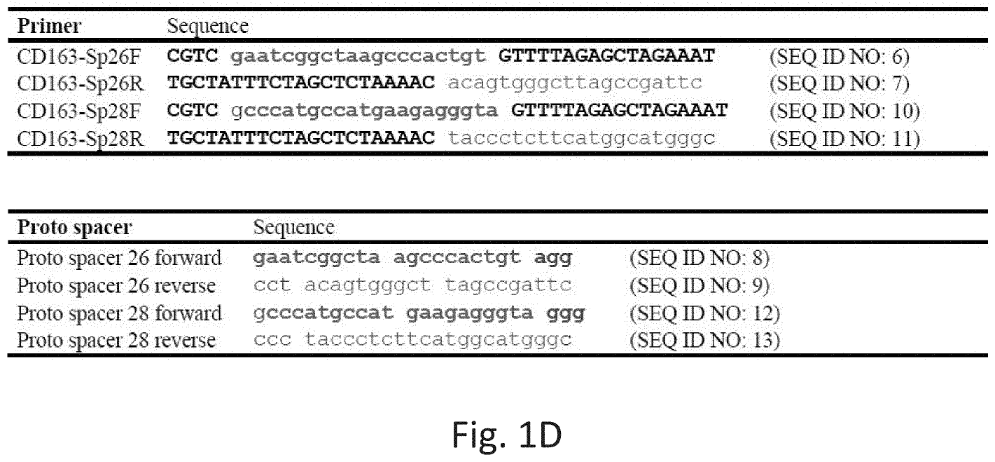

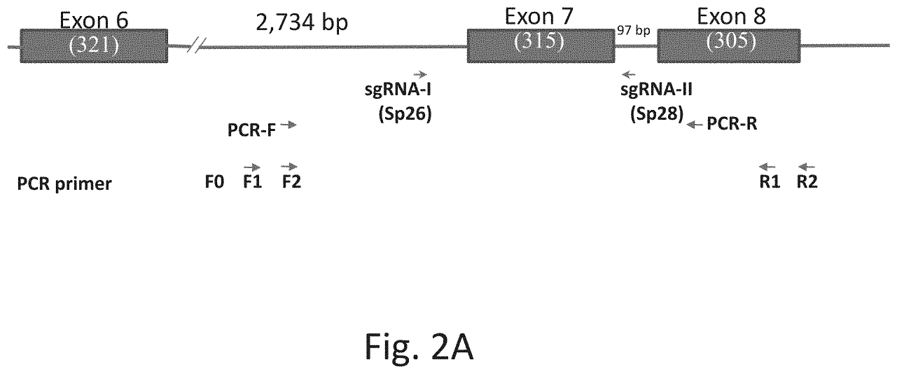

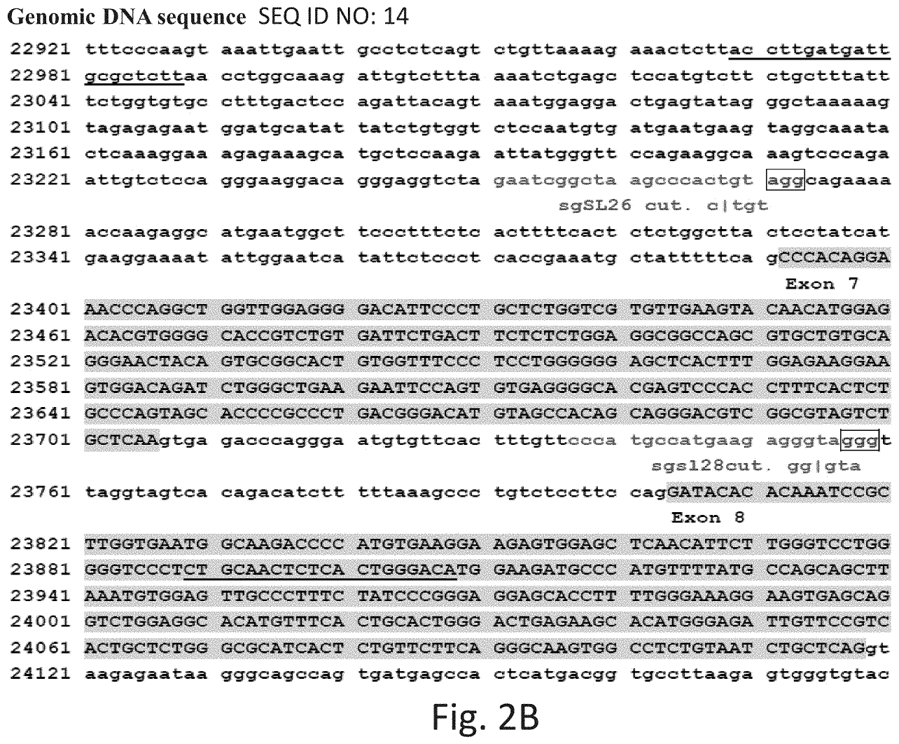

In some embodiments, a Cas9 endonuclease is used in a CRISPR method for making the gene-edited iPSCs as disclosed herein. The Cas9 enzyme may be one from Streptococcus pyogenes while other Cas9 homologs may also be used. Wild-type Cas9 or modified versions of Cas9 may be used. In some embodiments, Cas9 is modified to include two nuclear-localization signals (NLS) and a FLAG tag. The CRISPR technology involves the use of a genome-targeting nucleic acid that can direct the endonuclease to a specific target sequence within a target gene for gene editing at the specific target sequence. The genome-targeting nucleic acid can be an RNA. A genome-targeting RNA is described as a “guide RNA” or “gRNA” herein. A guide RNA comprises at least a spacer sequence which hybridizes to a target nucleic acid sequence within a target gene for editing, and a CRISPR repeat sequence. A spacer sequence in a gRNA is a sequence that defines the target sequence of a target gene of interest. In some embodiments, the spacer sequence is from 15 to 30 nucleotides in length, e.g. 15, 16, 17, 18, 19, 20, 21, 22, 23, 24, 25, 26, 27, 28, 29 or 30 nucleotides. In some embodiments, a spacer sequence contains 20 nucleotides. A target sequence is in a target gene that is adjacent to a PAM sequence and is the sequence to be modified by an RNA-guided nuclease e.g. Cas9. In a CRISPR/Cas system herein, the spacer sequence is designed to hybridize to a region of the target nucleic acid that is located 5′ of a PAM recognizable by a Cas9 enzyme used in the system. Each Cas9 enzyme has a specific PAM sequence that it recognizes in a target DNA. For example, S. pyogenes recognizes in a target nucleic acid a PAM that comprises the sequence 5′-NRG-3′, where R comprises A or G, where N is any nucleotide and N is immediately 3′ of the target nucleic acid sequence targeted by the spacer sequence. In some embodiments, the gRNAs disclosed herein target a CD163 gene, for example, target a site within exon 7 of a CD163 gene. Such a gRNA may comprise a spacer sequence complementary (complete or partially) to the target sequences within exon 7 of a CD163 gene. Exemplary target sequences in a CD163 gene and exemplary gRNAs specific to the CD163 gene are described in examples below (FIG. 1D and FIG. 2B). Delivery of an RNA-guided nuclease and gRNA, may be through direct injection or cell transfection using known methods, for example, electroporation or chemical transfection.

After simultaneous reprogramming and gene editing, gene-edited iPSC candidates are produced. Subcloning and genotyping are then carried out to obtain a gene-edited iPSC subclone having a genome with the gene edition of interest. Genotyping can be performed using any method known in the art, for example, an oligonucleotide ligase assay, restriction fragment length polymorphism (RFLP), polymerase chain reaction (PCR), sequencing, and immunoassay. In some embodiments, primers specific to the gene of interest are designed and used to determine the genotype of the gene in the gene-edited iPSC candidates.

The selected gene-edited iPSC subclones having a genome with the gene edition of interest are then transferred to an enucleated oocyte to generate a reconstituted embryo. In the present invention, an enucleated oocyte is the recipient cell in the nuclear transfer process. An oocyte is an immature female reproductive cell, one that has not completed the maturing process to form an ovum (gamete). The oocytes according to the present invention may be isolated from oviducts and/or ovaries of a mammal. In one embodiment, the oocytes are harvested by aspiration. Oocytes are typically matured in a variety of media known to a person skilled in the art prior to enucleation. The in vitro maturation of oocytes usually takes place in a maturation medium until the oocyte has reached the metaphase II stage or has extruded the first polar body. Enucleation of a matured oocyte may be performed by methods known in the art, for example, aspiration, physical removal, use of DNA-specific fluorochromes, exposure to ultraviolet light and/or chemically assisted enucleation. A gene-edited iPSC carrying given gene edition as a nuclear donor cell is injected into an enucleated oocyte as a receipt to form a reconstituted zygote. The zygotes are then activated with a chemical activator e.g. ionomycin or ethanol to form embryos. The embryos are cultured to the cleavage stage (2 to 4 cells) and then to the blastocyst stage. The cultured embryo is then transferred to a host mammal such that the embryo develops into a genetically edited non-human animal.

In a particular embodiment, the method of the present invention generates a CD163 gene editing pig by simultaneously transfecting porcine somatic cells with gene editing vectors and reprogramming vectors wherein the gene editing vectors provide gene edition of a CD163 gene to produce a plurality of CD163 gene-edited porcine iPSC (piPSC) candidates; subcloning and genotyping the gene-edited piPSC candidates to obtain a CD163 gene-edited piPSC subclone having a genome with the gene edition of the CD163 gene; transferring the CD163 gene-edited piPSC subclone into an enucleated porcine oocyte to generate a reconstituted porcine embryo; and culturing the reconstituted porcine embryo to reach the blastocyte stage to give rise to a pig with the gene edition of the CD163 gene. In certain examples, the gene edition of the CD163 gene is knock out or exon 7 deletion of the CD163 gene. The pig thus produced exhibits resistance to porcine reproductive respiratory syndrome virus (PRSV) infection. The method of the present invention also comprises the step of analyzing the chimeric pig for resistance to PRSV infection, and/or selecting a pig line from the plurality of gene-edited pigs as generated that exhibit resistance to PRSV as compared with a non gene-edited pig counterpart growing under the same conditions.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

The porcine fibroblasts were simultaneously treated by electroporation with the Yamanaka factors and CRISPR/Cas9 editing vectors to induce and achieve precisely gene deleted or knockout (KO) porcine pluripotent stem cells (piPSC) (CD163ΔE7 piPSC or KO piPSC). The piPSCs were subcloned and assessed their genome sequences to obtain precisely gene-edited piPSC. In this study, we have generated domestic pig CD163ΔE7 piPSC, and further used for somatic nuclear transfer (SCNT) to generate CD163ΔE7 blastocyst (BC) to be developed to pigs, which can resistant porcine reproductive respiratory syndrome virus (PRRSV) infection.

1. Material and Methods

1.1 Construction of Gene Editing Vectors

1.1.1 Cas9 vector

The coding region of the Streptococcus pyrogenes starin A20 cas9 protein was chemically synthesized with codons optimized for human. The 24th amino acid E (Glu) of the standard strain SF370 was replaced by D (Asp) in the A20 strain. A Daxx NLS was added to the C-terminus of the Cas9. The eukaryotic expression vector pCX-Flag₂-NLS₁-MCS-pA was used to build the pCX-Flag₂-NLS₁-Cas9-NLS₂ vector (first KO vector) [42] (FIG. 1A).

1.1.2 CD163 sgRNA vectors

The pig type III promoter, pU6, of RNA polymerase III corresponding to the mouse U6 promoter was cloned [43] and used to construct the ppU6-(BsaI)2-gRNA vector [42] (FIG. 1B) for expressing single-guide RNAs. In general, ppU6-(BsaI)2-gRNA vector including SEQ ID NO: 1 was recognized and cut by BsaI to produce two sticky-end overhangs. A primer pair with DNA sequence CGTCGN₁₉GTTTTAGAGCTAGAAAT (SEQ ID NO: 2) and TGCTATTTCTAGCTCTAAAACN₁₉C (SEQ ID NO: 3), where N₁₉ in SEQ ID NO: 2 and N₁₉ SEQ ID NO: 3 are complementary to each other, were annealed and ligated with the BsaI restriction enzyme treated ppU6-(BsaI)2-gRNA vector to create the ppU6-Sp-gRNA single-guide RNA expression vector (FIG. 1C, including a strand of SEQ ID NO: 4 and its complementary strand of SEQ ID NO: 5). Specifically, to construct CD163 sgRNA vectors, pCD163-Sp26F (SEQ ID NO: 6) and pCD163-Sp26R (SEQ ID NO: 7), as the first primer pair, were annealed to each other and ligated with the BsaI restriction enzyme treated ppU6-(BsaI)2-gRNA vector to produce ppU6-pCD163Sp26-gRNA vector; and pCD163-Sp28F (SEQ ID NO: 10) and pCD163-Sp28R (SEQ ID NO: 11), as the second primer pair, were also annealed to each other and ligated with the BsaI restriction enzyme treated ppU6-(BsaI)2-gRNA vector to produce ppU6-pCD163Sp28-gRNA vector (FIG. 1D). The proto spacer 26 includes a strand of SEQ ID NO: 8, its complementary strand being of SEQ ID NO: 9 (FIG. 1D). The proto spacer 28 includes a strand of SEQ ID NO: 12, its complementary strand being SEQ ID NO: 13 (FIG. 1D).

In the genomic DNA of CD163 (SEQ ID NO: 14), the DNA fragment between proto-spacer 26 and proto-spacer 28 can be removed simply by co-transfection of the vectors including pCX-Flag₂-NLS₁-Cas9-NLS₂, ppU6-pCD163Sp26-gRNA and ppU6-pCD163Sp28-gRNA together. The Cas9-sgRNA complexes can recognize the proto spacer sequences of Sp26 and Sp28 and cut porcine genomic DNA at “sgSL26 cut” and “sgSL28 cut” respectively (FIG. 2A and FIG. 2B) to remove CD163 exon 7. As a result, cells with CD163 exon 7 edited/deleted (CD163ΔE7) genotype can be produced.

1.2 Construction of Reprogramming Vectors

The iPSC inducing vectors for reprograming, pCX-Oct4-2A-Sox2-2A-K1f4-2A-NANOG (pCX-OSKN), pCX-cMyc and pCX-Tag, were produced based on the previously described vectors [16] where the reprogramming genes were native for pig Oct4, pig Sox2 and SV40 large T-antigen (Tag) and codon-optimized for pig Klf4, pig c-Myc and human NANOG.

1.3 Treatments with Combination of iPSC Inducing and Gene Editing

1.3.1 Establishment of Primary Fibroblast Cells

The ear tissues were cut from the new born piglet, breeding boar or sow and stored in Dulbecco's phosphate-buffered saline (D-PBS) with 10× penicillin/streptomycin/amphotericin (PSA; CORNING, USA). The tissues were packed with ice during transported back to laboratory. After cutting off surface hair, the ear tissues were sterilized by 75% ethanol and intensively washed twice by PBS with 3 xPSA. The tissues were cut to small pieces by scissors and 50-60 μL of tissue suspension was seeded into 6 cm dish. The dish was covered with cover-glass to enhance the tissues attachment on the dish, and finally 5 mL DMEM with 10xPSA were supplied for further cultivation. Each tissue sample was seeded into 10-15 dishes in total. During 7-14 days cultivation, the contaminated dishes, of which medium turned yellow, were discarded and those dishes with the primary fibroblast out-grow well around the tissues were dissociated with trypsin and re-seeded to 10 cm dish to amplify the fibroblast for further usages, e.g. for induction/infection to generate gene-edited piPSC or cryopreservation.

1.3.2 Simultaneous Induction of Porcine iPSCs and Gene Editing of CD163 Exon 7 Deletion (AE7)

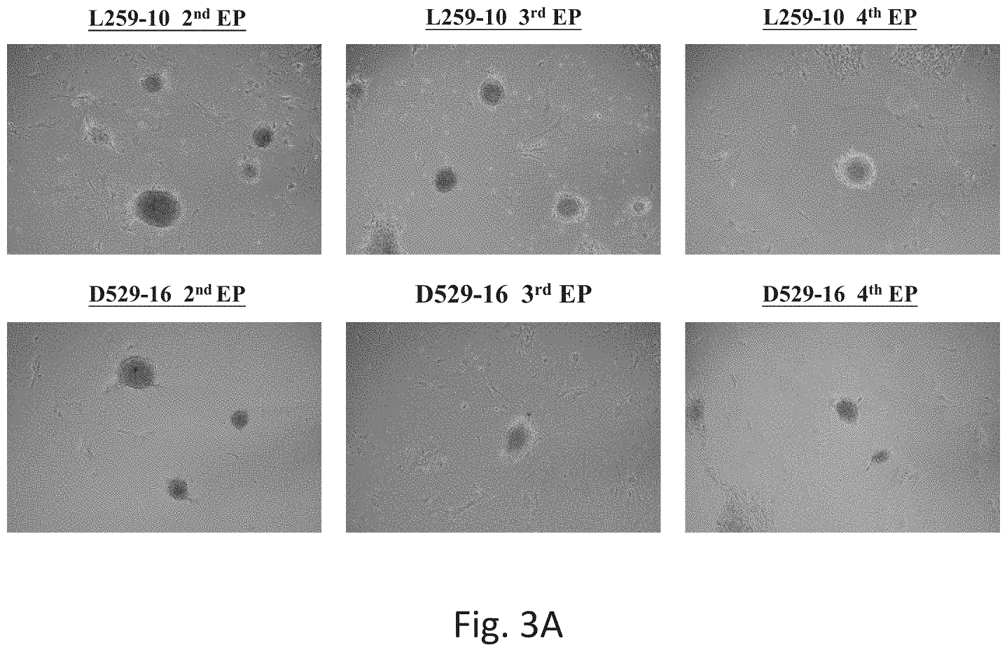

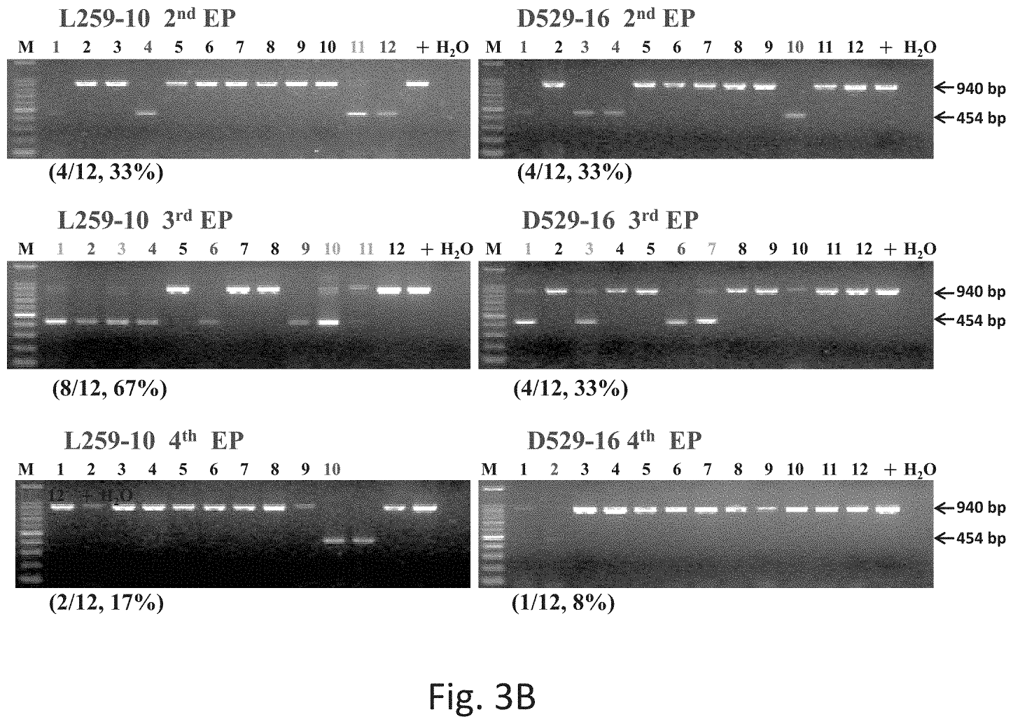

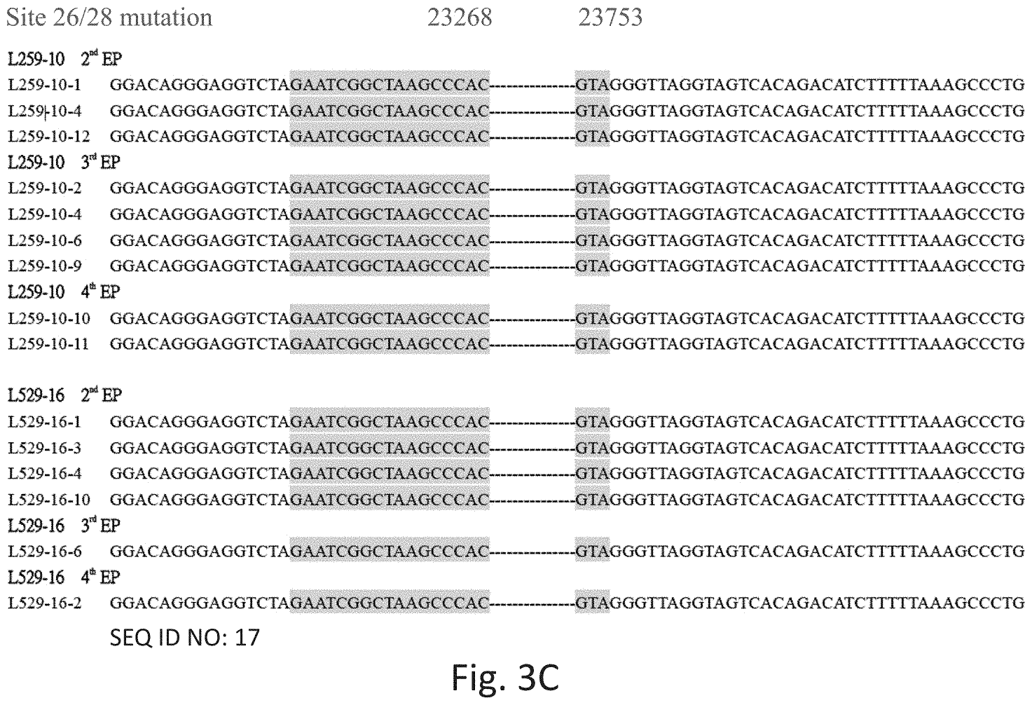

The reprograming vectors and gene-editing vectors were simultaneously transfected into the primary fibroblast cells. The reprograming vectors include pCX-Oct4-2A-Sox2-2A-K1f4-2A-NANOG (pCX-OSKN), pCX-cMyc and pCX-TAg at a weight ratio of 2:1:1 (5.0, 2.5 and 2.5 μg/100 μL). The gene-editing vectors include ppU6-pCD163-Sp26-sgRNA, ppU6-pCD163-Sp28-sgRNA and pCX-Flag₂-NLS₁-Cas9-NLS₂ at a weight ratio of 1:1:2 (2.5, 2.5 and 5.0 μg/100 μL). The transfection by electroporation (EP) was conducted by using Neon transfection system (Gibcoo, USA) with 1×106/100 μL cells suspension. After one 30 μsec 1.2 kV/cm AC pulse, the cells were cultured in Dulbecco's Modified Eagle Medium (DMEM) containing 10% Fetal Bovine Serum (FBS), 1×GlutaMAX, 1×nonessential amino acid, 1×β-mercaptoethanol, 1×penicillin/streptomycin, 50 μg/mL Vit. C and 5.25 μg/mL Cellmaxin. After 7-day cultivation, the 2nd EP and cultivation was conducted with the same conditions as in 1^{st} EP except the cells were cultivated with mouse embryonic fibroblast (MEF) Feeder. Around 7 days following 2^{nd} EP, the candidate piPSC appeared and were picked up by glass mouth pipet, and the remaining cells further underwent 3^{rd} and/or 4th EP with conditions the same as the 2nd EP. The picked-up candidate piPSC clones (FIG. 3A) were sub-cloned for further gene assessment.

1.3.3 The Assessment of Gene-Edited piPSCs

The candidate piPSC clones were picked-out, dispersed in 10 μL accutase (CORNING, USA) for 5 min and disassociated by glass mouth pipette. After seeding in 24 well culture dish on MEF-feeder, the candidates sub-clones proliferated to 80 to 90% confluent and then again were treated by the same procedures and cultivated in 0.2% gelatin-coated (feeder cell free) 12 well dish. When the cells growth reach to about 90% confluent, the candidates were harvested for genomic DNA extraction and assessment. The genomic DNA were amplified by PCR with F2 (SEQ ID NO:15) and R2 (SEQ ID NO:16) primers.

Primer Seq. (5′→3′)
F2
(SEQ ID NO: 15)
ACTTCTCTTTGGGACTGCAAGAATTGGCAG
R2
(SEQ ID NO: 16)
AGGCGAAGTTGACCACTCCCTGCAAA

The PCR amplicon of genomic DNA for wild type and CD163ΔE7 were 940 and 454 bp, respectively. Those homologous edited sub-clones were further analyzed by PCR products sequencing and the homologous sub-clones were proliferated for further usage or storage.

1.4 Somatic Cell Nuclear Transfer (SCNT)

1.4.1 Collection of Porcine Oocytes

The porcine ovaries were collected from slaughterhouse, kept in normal saline at 30-35° C. and sent back to laboratory within 2 hours. The cumulus-oocytes complexes (COCs) were harvested from 3 to 6 mm follicles by 18 gauge needle/syringe. The COCs at least with 3 layers cumulus cells were collected and washed with medium for further in vitro maturation (IVM).

1.4.2 Porcine COCs in IVM

The IVM medium is composed of medium 199 (M199) and addition of 10 IU/mL pregnant Mare's serum gonadotropin (PMSG), 10 IU/mL human chorionic gonadotrophin (hCG), 20 ng/mL epidermal growth factor (EGF), 20 ng/mL non-essential amino acid (AA), 10% porcine follicular fluid (pFF), 100 μg/mL cysteine (ComaDex, USA) and 50 μg/mL Vit. C. After cultivation in the IVM medium for 20 to 22 hours, the COCs were moved into same IVM medium without hormone for cultivation for another 20 to 22 hours. All the cumulus cells expending COCs were removed and those oocytes with first polar body were selected for somatic cell nuclear transfer (SCNT).

1.4.3 SCNT

All IVM oocytes were chemically enucleated and CD163ΔE7 piPSC was directly injected into the cytoplasm of the oocytes. After reconstitution, the zygotes were chemically activated with 15 μM ionomycin for 5 min and followed with 5 μM TPEN [N, N, N′, N′-tetrakis (2-pyridinylmethyl)-1, 2-ethanediamine] for another 15 min. All reconstituted embryos were further cultivated in porcine zygote medium 5 (PZMS) with 500 nM scriptaid for 16 h to reprogram and enhance the endogenous gene expression. Followed, all reconstituted embryos were cultured in PZM-5 with 50 μg/mL Vit. C and 10 nM melatonin for 48 h to reach cleavage stages (2-4 cells) and for 6 to 7 days to reach blastocyst stage.

2. Results

2.1 Generation and Analysis of Porcine CD163 Gene Exon 7 Deleted (CD163ΔE7) piPSC

After 2^{nd} to 4th EP, typical iPSCs with dome morphology have been achieved from pigs L259-10 and D529-16 (FIG. 3A). The iPSCs candidates were further sub-cloned and PCR was performed for genotypes analysis. The results show that gene-edited piPSC subclones, including homogenous exon 7 deletion in both alleles and heterologous exon 7 deletion in only one of the two alleles, were obtained via 2^{nd} to 4th EP (FIG. 3B). These subclones were further analyzed by PCR amplicon sequencing and the subclones L-259-10-1, L-259-10-4 and L-259-10-12 (2^{nd} EP), L259-10-2, L259-10-4, L259-10-6 and L259-10-9 (3^{nd} EP), L259-10-10 and L259-10-11 (4^{nd} EP), L529-16-1, L529-16-3, L529-16-4 and L529-16-10 (2^{nd} EP), L529-16-6 (3^{nd} EP) and L529-16-2 (411d EP) (FIG. 3C) were confirm to have deletion of exon 7 (from 23268 bp to 23753 bp). These CD163ΔE7 piPSC were used for SCNT to generate reconstituted embryos.

2.2 Generation of CD163ΔE7 Blastocysts

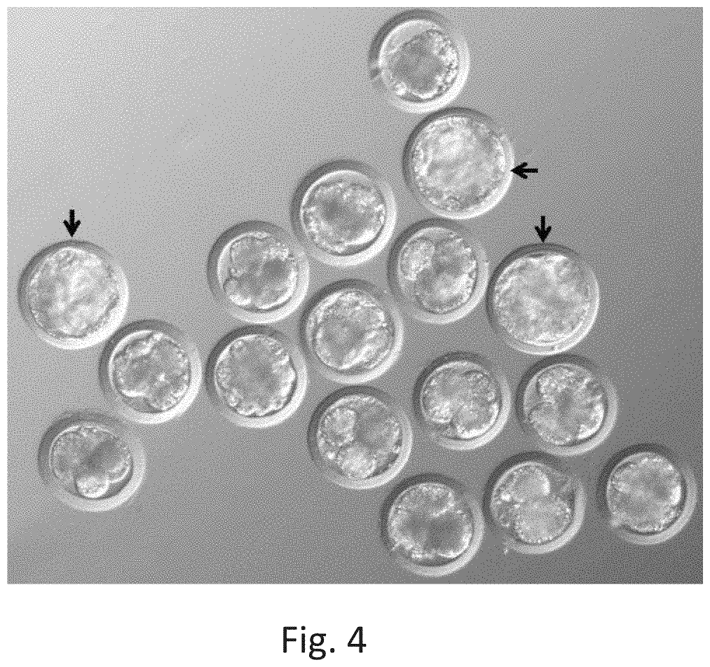

The CD163ΔE7 piPSCs as nuclear donors were microinjected into the cytoplasm of the enucleated matured oocytes in IVM. The resultant reconstituted zygotes were further cultivated in PZMS for 6 to 7 days and then the CD163ΔE7 blastocysts were obtained (FIG. 4) which can be transferred to a recipient female's uterus to develop into a genetically edited pig.

3. Conclusions

In this study, gene edited piPSCs candidates were generated by co-transfection of pCX-pOct4-2A-pSox2-2A-pK1f4-2A-hNANOG (pCX-OSKN), pCX-pcMyc and pCX-TAg plasmid vectors combined with sgRNA plasmid vectors and a Cas9 plasmid vector; theses candidates were subcloned and screened by PCR and sequencing at in vitro cell stage to obtain precisely CD163 gene-edited piPSCs, which were used as a nuclear donor in SCNT so as to generate CD163ΔE7 blastocysts and pigs which are capable of resisting PRRSV infection. This instant method can save the time and reduce the cost of breeding program especially on large domestic animals, for instance via CD163 exon 7 alleles deleted or CD163 KO, PRRSV resistance pigs can generated.

### REFERENCES

1. Takahashi K, Yamanaka S. 2006. Induction of pluripotent stem cells from mouse embryonic and adult fibroblast cultures by defined factors. Cell 126:663-676. https://doi: 10.1016/j.ce11.2006.07.024.
2. Takahashi K, Tanabe K, Ohnuki M, et al. 2007. Induction of pluripotent stem cells from adult human fibroblasts by defined factors. Cell 131:861-872. https://doi: 10.1016/j.ce11.2007.11.019
3. Yu J, Vodyanik M A, Smuga-Otto K, et al. 2007. Induced plur-ipotent stem cell lines derived from human somatic cells. Science 318:1917-1920. https://doi: 10.1126/science.1151526
4. Esteban M A, Xu J, Yang J, et al. 2009. Generation of induced pluripotent stem cell lines from Tibetan miniature pig. J. Biol. Chem. 284:17634-17640. https://doi: 10.1074/jbc.M109.008938.
5. Ezashi T, Telugu B P, Alexenko A P, et al. 2009. Derivation of induced pluripotent stem cells from pig somatic cells. Proc. Natl. Acad. Sci. USA 106:10993-10998. https://doi: 10.1073/pnas.0905284106.
6. Wu Z, Chen J, Ren J, et al. 2009. Generation of pig induced pluripotent stem cells with a drug-inducible system. J. Mol. Cell Biol. 1:46-54. https://doi: 10.1093/jmcb/mjp003.
7. West F D, Terlouw S L, Kwon D J, et al. 2010. Porcine induced pluripotent stem cells produce chimeric offspring. Stem Cells Dev. 19:1211-1220. https://doi: 10.1089/scd.2009.0458.
8. West F D, Uhl E W, Liu Y, et al. 2011. Brief report: chimeric pigs produced from induced pluripotent stem cells demonstrate germline transmission and no evidence of tumor formation in young pigs. Stem Cells 29:1640-1643. https://doi: 10.1002/stem.713
9. Liu K, Ji G, Mao J, et al. 2012. Generation of porcine-induced pluripotent stem cells by using OCT4 and KLF4 porcine factors. Cell Reprogram. 14:505-513. https://doi: 10.1089/ce11.2012.0047.
10. Du X, Feng T, Yu D, et al. 2015. Barriers for deriving transgene-free pig iPS cells with episomal vectors. Stem Cells 33:3228-3238. https://doi: 10.1002/stem.2089.
11. Cheng D, Guo Y, Li Z, et al. 2012. Porcine induced pluripotent stem cells require LIF and maintain their developmental potential in early stage of embryos. PLoS One 7:e51778. https://doi: 10.1371/journal.pone.0051778.
12. Yuan Y, Lee K, Park K W, et al. 2014. Cell cycle synchronization of leukemia inhibitory factor (LIF)-dependent porcine-induced pluripotent stem cells and the generation of cloned embryos. Cell Cycle 13:1265-1276. https://doi: 10.4161/cc.28176.
13. Kim E, Hwang S U, Yoo H, et al. 2016. Putative embryonic stem cells derived from porcine cloned blastocysts using induced pluripotent stem cells as donors. Theriogenology 85:601-616. https://doi: 10.1016/j.theriogenology.2015.09.051.
14. Fan N, Chen J, Shang Z, et al. 2013. Piglets cloned from induced pluripotent stem cells. Cell Res. 23:162-166. https://doi: 10.1038/cr.2012.176
15. Yu D, Wang J, Zou H, et al. 2018. Silencing of retrotransposon-derived imprinted gene RTL1 is the main cause for postimplantational failures in mammalian cloning. Proc. Natl. Acad. Sci. USA 115:E11071-E11080. https://doi: 10.1073/pnas.1814514115.
16. Chen C H, Su Y H, Lee K H, Chuang C K. 2016. Germline competent pluripotent mouse stem cells generated by plasmid vectors. Anim. Biotechnol. 27:157-165. https://doi: 10.1080/10495398.2016.1140056.
17. Moehle E A, Rock J M, Lee Y L, et al. 2007. Targeted gene addition into a specified location in the human genome using designed zinc finger nucleases. Proc. Natl. Acad. Sci. USA. 104:3055-3060. https://doi: 10.1073/pnas.0611478104
18. Christian M, Cermak T, Doyle E L, et al. 2010. Targeting DNA double-strand breaks with TAL effector nucleases. Genetics 86:757-761. https://doi:10.1534/genetics.110.120717
19. Cong L, Ran F A, Cox D, et al. 2013. Multiplex genome engineering using CRISPR/Cas systems. Science 339:819-923. https://doi: 10.1126/science.1231143
20. Chuang C K, Lin W M. 2021. Points of view on the tools for genome/gene editing. Int. J. Mol. Sci. 22:9872. https://doi: 10.3390/ijms22189872.
21. Howden S E, Maufort J P, Duffin B M, Elefanty A G, Stanley E G, Thomson J A. 2015. Simultaneous Reprogramming and Gene Correction of Patient Fibroblasts. Stem Cell Reports 5:1109-1118. https://doi: 10.1016/j.stemcr.2015.10.009.
22. Wang P, Lin M, Pedrosa E, et al. 2015. CRISPR/Cas9-mediated heterozygous knockout of the autism gene CHD8 and characterization of its transcriptional networks in neurodevelopment. Mol. Autism. 6:55. https://doi: 10.1186/s13229-015-0048-6. eCollection 2015.
23. Kim B Y, Jeong S, Lee S Y, et al. 2016. Concurrent progress of reprogramming and gene correction to overcome therapeutic limitation of mutant ALK2-iPSC. Exp. Mol. Med. 48:e237. https://doi: 10.1038/emm.2016.43.
24. Bell S, Peng H, Crapper L, et al. 2017. A rapid pipeline to model rare neurodevelopmental disorders with simultaneous CRISPR/Cas9 gene editing. Stem Cells Transl. Med. 26:886-896. https://doi: 10.1002/sctm.16-0158.
25. Tidball A M, Dang L T, Glenn T W, et al. 2017. Rapid generation of human genetic loss-of-function iPSC lines by simultaneous reprogramming and gene editing. Stem Cell Reports 9:725-731. https://doi: 10.1016/j.stemcr.2017.07.003.
26. Melo U S, de Souza Leite F, Costa S, Rosenberg C, Zatz M. 2018. A fast method to reprogram and CRISPR/Cas9 gene editing from erythroblasts. Stem Cell Res. 31:52-54. https://doi: 10.1016/j.scr.2018.07.002.
27. Wen W, Cheng X, Fu Y, et al. 2018. High-level precise knockin of iPSCs by simultaneous reprogramming and genome editing of human peripheral blood mononuclear cells. Stem Cell Reports 10:1821-1834. https://doi: 10.1016/j.stemcr.2018.04.013.
28. Howden S, Hosseini Far H, Motazedian A, et al. 2019. The use of simultaneous reprogramming and gene correction to generate an osteogenesis imperfecta patient COL1A1 c. 3936 G>T iPSC line and an isogenic control iPSC line. Stem Cell Res. 38:101453. https://doi: 10.1016/j.scr.2019.101453.
29. Ye T, Duan Y, Tsang H W S, et al. 2021. Efficient manipulation of gene dosage in human iPSCs using CRISPR/Cas9 nickases. Commun. Biol. 4:195. https://doi: 10.1038/s42003-021-01722-0.
30. Van Breedam W, Delputte P L, Van Gorp H, et al. 2010. Porcine reproductive and respiratory syndrome virus entry into the porcine macrophage. J. Gen. Virol. 91(Pt 7):1659-1667. https://doi:10.1099/vir.0.020503-0
31. Zhang Q, Yoo D. 2015. PRRS virus receptors and their role for pathogenesis. Vet. Microbiol. 177:229-241. https://doi: 10.1016/j.vetmic.2015.04.002
32. Whitworth K M, Lee K, Benne J A, et al. 2014. Use of the CRISPR/Cas9 system to produce genetically engineered pigs from in vitro-derived oocytes and embryos. Biol. Reprod. 91:78. https://doi:10.1095/biolreprod.114.121723
33. Yang H, Zhang J, Zhang X, et al. 2018. CD163 knockout pigs are fully resistant to highly pathogenic porcine reproductive and respiratory syndrome virus. Antiviral Res. 151:63-70. https://doi: 10.1016/j.antiviral. 2018.01.004
34. Xu K, Zhou Y, Mu Y, et al. 2020. CD163 and pAPN double-knockout pigs are resistant to PRRSV and TGEV and exhibit decreased susceptibility to PDCoV while maintaining normal production performance. Elife 9:e57132. https://doi:10.7554/eLife.57132
35. Tanihara F, Hirata M, Nguyen N T, et al. 2021. Generation of CD163-edited pig via electroporation of the CRISPR/Cas9 system into porcine in vitro-fertilized zygotes. Anim. Biotechnol. 32: 147-154. https://doi: 10.1080/10495398.2019.1668801
36. Burkard C, Lillico S G, Reid E, et al. 2017. Precision engineering for PRRSV resistance in pigs: Macrophages from genome edited pigs lacking CD163 SRCRS domain are fully resistant to both PRRSV genotypes while maintaining biological function. PLoS Pathogens 13: e1006206. https://DOI: 10.1371/journal.ppat.1006206
37. Wang H, Shen L, Chen J, et al. 2019. Deletion of CD163 exon 7 confers resistance to highly pathogenic porcine reproductive and respiratory viruses on pigs. Int. J. Biol. Sci. 15:1993-2005. https://doi:10.7150/ijbs.34269. eCollection 2019
38. Guo C, Wang M, Zhu Z, et al. 2019. Highly efficient generation of pigs harboring a partial deletion of the CD163 SRCRS domain, which are fully resistant to porcine reproductive and respiratory syndrome virus 2 infection. Front. Immunol. 10:1846. https://doi.org/10.3389/fimmu.2019.01846
39. Whitworth K M, Rowland R R R, Ewen C L, et al. 2016. Gene-edited pigs are protected from porcine reproductive and respiratory syndrome virus. Nat. Biotech. 34:20-22. https://doi: 10.1038/nbt.3434
40. Burkard C, Opriessnig T, Mileham A J, et al. 2018. Pigs lacking the scavenger receptor cysteine-rich domain 5 of CD163 are resistant to porcine reproductive and respiratory syndrome virus 1 infection. J. Virol. 92:pii: e00415-18. https://doi:10.1128/JVI.00415-18.
41. Whitworth K M, Prather R S. 2017. Gene editing as applied to prevention of reproductive porcine reproductive and respiratory syndrome. Mol. Reprod. Dev. 84:926-3. https://doi: 10.1002/mrd.22811.
42. Su Y H, Lin T Y, Huang C L, Tu C F, Chuang C K. 2015. Construction of a CRISPR-Cas9 system for pig genome targeting. Anim. Biotechnol. 26:279-288. https://doi: 10.1080/10495398.2015.1027774.
43. Chuang C K, Lee K H, Fan C T, Su Y S. 2009. Porcine type III RNA polymerase III promoters for short hairpin RNA expression. Anim. Biotechnol. 20:34-39. https://doi: 10.1080/10495390802603064.

What is claimed is:

1. A method of producing a genetically edited non-human embryo and/or a resultant genetically edited non-human animal, comprising
(a) gene editing non-human mammalian somatic cells to induce a gene edition of interest and simultaneously reprograming the cells to reprogram into induced pluripotency stem cells (iPSCs), so as to produce a plurality of gene-edited iPSC candidates;
(b) subcloning and genotyping the gene-edited iPSC candidates to obtain a gene-edited iPSC subclone having a genome with the gene edition of interest;
(c) transferring the gene-edited iPSC subclone into an enucleated oocyte to generate a reconstituted embryo; and
(d) culturing the reconstituted embryo to reach the blastocyst stage to give rise to a non-human gene-edited animal with the gene edition of interest.

2. The method of claim 1, wherein the gene editing is CRISPR/Cas9-based gene editing.

3. The method of claim 1, wherein the reprogramming factors comprises Klf4, c-Myc, Nanog, Oct4, Sox2 and SV40 large T antigen.

4. The method of claim 1, wherein the somatic cells are simultaneously transfected by CRISPR/Cas9-based gene editing vectors and reprogramming vectors.

5. The method of claim 4, wherein the CRISPR/Cas9-based gene editing vectors comprise
a Cas9 vector comprising nucleic acids encoding a Cas9 protein, and
one or more gRNA vectors each comprising nucleic acids encoding a gRNA molecule for targeting Cas9 to a gene of interest to induce the gene edition.

6. The method of claim 5, wherein the reprogramming vectors comprise
a first reprogramming vector comprising nucleic acids encoding Oct4, Sox2, Klf4 and Nanog;
a second reprogramming vector comprising nucleic acids encoding c-Myc; and
a third reprogramming vector comprising nucleic acids encoding SV40 large T antigen.

7. The method of claim 1, wherein the somatic cells and the oocyte are from the same species.

8. The method of claim 1, wherein the somatic cells are fibroblasts.

9. The method of claim 1, wherein the gene edition is gene knock-in or gene knock-out or partial deletion.

10. The method of claim 1, wherein the non-human animal is selected from the group consisting of sheep, cattle, deer, goat, monkeys, camels and pigs.

11. The method of claim 1, wherein the non-human animal is a pig.

12. The method of claim 11, wherein the gene edition is gene knock-out or partial deletion of CD163.

13. A method of providing a CD163 gene-edited pig, comprising
(a) simultaneously transfecting porcine somatic cells with gene editing vectors and reprogramming vectors, wherein the gene editing vectors provide gene edition of a CD163 gene, so as to produce a plurality of gene-edited porcine iPSC (piPSC) candidates,
(b) subcloning and genotyping the gene-edited piPSC candidates to obtain a CD163 gene-edited piPSC subclone having a genome with the gene edition of the CD163 gene;
(c) transferring the CD163 gene-edited piPSC subclone into an enucleated porcine oocyte to generate a reconstituted porcine embryo; and
(d) culturing the reconstituted porcine embryo to reach the balstocyst stage to give rise to a CD163 gene-edited pig with the gene edition of the CD163 gene.

14. The method of claim 13, wherein the gene editing vectors are CRISPR/Cas9-based gene editing vectors.

15. The method of claim 13, wherein the reprogramming vectors encode one or more reprogramming factors selected from the group consisting of Klf4, c-Myc, Nanog, Oct4, Sox2 and SV40 large T antigen.

16. The method of claim 15, wherein the CRISPR/Cas9-based gene editing vectors comprise
a Cas9 vector comprising nucleic acids encoding a Cas9 protein, and
one or more gRNA vectors each comprising nucleic acids encoding a gRNA molecule for targeting Cas9 to the CD163 gene to induce gene knock-out or partial deletion of the CD163 gene.

17. The method of claim 16, wherein the reprogramming vectors comprise
a first reprogramming vector comprising nucleic acids encoding Oct4, Sox2, Klf4 and Nanog;
a second reprogramming vector comprising nucleic acids encoding c-Myc; and
a third reprogramming vector comprising nucleic acids encoding SV40 large T antigen.

18. The method of claim 13, wherein porcine somatic cells are porcine fibroblasts.

19. The method of claim 13, wherein the gene edition of the CD163 gene is knock out or exon 7 deletion of the CD163 gene.

20. The method of claim 13, wherein the gene-edited pig exhibits resistance to porcine reproductive respiratory syndrome virus (PRSV) infection.

21. A method to provide resistance to porcine reproductive respiratory syndrome virus (PRSV) infection in a pig, comprising
(a) simultaneously transfecting porcine somatic cells with gene editing vectors and reprogramming vectors, wherein the gene editing vectors provide gene knockout or partial deletion of a CD163 gene, so as to produce a plurality of gene-edited porcine iPSC (piPSC) candidates;
(b) subcloning and genotyping the gene-edited piPSC candidates to obtain a population of CD163 gene-edited piPSC subclones having a genome with gene knockout or partial deletion of the CD163 gene;
(c) transferring each of the CD163 gene-edited iPSC subclones into an enucleated porcine oocyte to generate reconstituted porcine embryos;
(d) culturing the reconstituted porcine embryos to reach the blastocyte stage to give rise to a plurality of CD163 gene-edited pigs; and
(e) selecting a pig line from the plurality of CD163 gene-edited pigs generated in (d) that exhibits resistance to PRSV as compared with a non gene-edited pig counterpart growing under the same conditions


SEQUENCE LISTING (20 sequences)

SEQ ID NO: 1 - DNA, 467 bp, synthetic construct
  source 1..467: mol_type = other DNA
ggatccaggaggactccagggacaaccgctgggctcgcagacccgcaatagtgagagaagcgctaggggaacgagcaccacgtgaccgagcgtgacagtaaggcgcgcggaggtcgggcgggcagagggccaatttcccatggtcccttcatttgcatataatatgtaggaaggttaaagagacaattctacttaactctcccaaaatcacaaagatatgatgacagtatgtgtgacatagaaggtcataaatcctcgaatacttttaaattgaaaaaaattactcttctatagtcaccataatcaaaaaagtttttatgttcttggctttatataccctctgagaagacccagccgtcggagaccgcggccgcacgcgtctcgaggtctctagcaagttaaaataaggctagtccgttatcaacttgaaaaagtggcaccgagtcggtgctttttttagatctaga

SEQ ID NO: 2 - DNA, 41 bp, synthetic construct
  source 1..41: mol_type = other DNA
cgtcgnnnnnnnnnnnnnnnnnnngttttagagctagaaat

SEQ ID NO: 3 - DNA, 41 bp, synthetic construct
  source 1..41: mol_type = other DNA
tgctatttctagctctaaaacnnnnnnnnnnnnnnnnnnnc

SEQ ID NO: 4 - DNA, 54 bp, synthetic construct
  source 1..54: mol_type = other DNA
agacccagccgtcgnnnnnnnnnnnnnnnnnnngttttagagctagaaatagca

SEQ ID NO: 5 - DNA, 54 bp, synthetic construct
  source 1..54: mol_type = other DNA
tgctatttctagctctaaaacnnnnnnnnnnnnnnnnnnncgacggctgggtct

SEQ ID NO: 6 - DNA, 41 bp, synthetic construct
  source 1..41: mol_type = other DNA
cgtcgaatcggctaagcccactgtgttttagagctagaaat

SEQ ID NO: 7 - DNA, 41 bp, synthetic construct
  source 1..41: mol_type = other DNA
tgctatttctagctctaaaacacagtgggcttagccgattc

SEQ ID NO: 8 - DNA, 23 bp, synthetic construct
  source 1..23: mol_type = other DNA
gaatcggctaagcccactgtagg

SEQ ID NO: 9 - DNA, 23 bp, synthetic construct
  source 1..23: mol_type = other DNA
cctacagtgggcttagccgattc

SEQ ID NO: 10 - DNA, 42 bp, synthetic construct
  source 1..42: mol_type = other DNA
cgtcgcccatgccatgaagagggtagttttagagctagaaat

SEQ ID NO: 11 - DNA, 42 bp, synthetic construct
  source 1..42: mol_type = other DNA
tgctatttctagctctaaaactaccctcttcatggcatgggc

SEQ ID NO: 12 - DNA, 42 bp, synthetic construct; identical to SEQ ID NO: 10
  source 1..42: mol_type = other DNA

SEQ ID NO: 13 - DNA, 42 bp, synthetic construct; identical to SEQ ID NO: 11
  source 1..42: mol_type = other DNA

SEQ ID NO: 14 - DNA, 1260 bp, Sus scrofa
  source 1..1260: mol_type = genomic DNA
tttcccaagtaaattgaattgcctctcagtctgttaaaagaaactcttaccttgatgattgcgctcttaacctggcaaagattgtctttaaaatctgagctccatgtcttctgctttatttctggtgtgcctttgactccagattacagtaaatggaggactgagtatagggctaaaaagtagagagaatggatgcatattatctgtggtctccaatgtgatgaatgaagtaggcaaatactcaaaggaaagagaaagcatgctccaagaattatgggttccagaaggcaaagtcccagaattgtctccagggaaggacagggaggtctagaatcggctaagcccactgtaggcagaaaaaccaagaggcatgaatggcttccctttctcacttttcactctctggcttactcctatcatgaaggaaaatattggaatcatattctccctcaccgaaatgctatttttcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagtgagacccagggaatgtgttcactttgttcccatgccatgaagagggtagggttaggtagtcacagacatctttttaaagccctgtctccttccaggatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcaggtaagagaataagggcagccagtgatgagccactcatgacggtgccttaagagtgggtgtac

SEQ ID NO: 15 - DNA, 30 bp, synthetic construct
  source 1..30: mol_type = other DNA
acttctctttgggactgcaagaattggcag

SEQ ID NO: 16 - DNA, 26 bp, synthetic construct
  source 1..26: mol_type = other DNA
aggcgaagttgaccactccctgcaaa

SEQ ID NO: 17 - DNA, 71 bp, synthetic construct
  source 1..71: mol_type = other DNA
ggacagggaggtctagaatcggctaagcccacgtagggttaggtagtcacagacatctttttaaagccctg

SEQ ID NO: 18 - DNA, 13 bp, synthetic construct
  source 1..13: mol_type = other DNA
gacggctgggtct

SEQ ID NO: 19 - protein, 1110 aa, Sus scrofa
MVLLEDSGSADFRRCSAHLSSFTFAVVAVLSACLVTSSLGGKDKELRLTGGENKCSGRVEVKVQEEWGTVCNNGWDMDVVSVVCRQLGCPTAIKATGWANFSAGSGRIWMDHVSCRGNESALWDCKHDGWGKHNCTHQQDAGVTCSDGSDLEMRLVNGGNRCLGRIEVKFQERWGTVCDDNFNINHASVVCKQLECGSAVSFSGSANFGEGSGPIWFDDLVCNGNESALWNCKHEGWGKHNCDHAEDAGVICLNGADLKLRVVDGLTECSGRLEVKFQGEWGTICDDGWDSDDAAVACKQLGCPTAVTAIGRVNASEGTGHIWLDSVSCHGHESALWQCRHHEWGKHYCNHNEDAGVTCSDGSDLELRLKGGGSHCAGTVEVEIQKLVGKVCDRSWGLKEADVVCRQLGCGSALKTSYQVYSKTKATNTWLFVSSCNGNETSLWDCKNWQWGGLSCDHYDEAKITCSAHRKPRLVGGDIPCSGRVEVQHGDTWGTVCDSDFSLEAASVLCRELQCGTVVSLLGGAHFGEGSGQIWAEEFQCEGHESHLSLCPVAPRPDGTCSHSRDVGVVCSRYTQIRLVNGKTPCEGRVELNILGSWGSLCNSHWDMEDAHVLCQQLKCGVALSIPGGAPFGKGSEQVWRHMFHCTGTEKHMGDCSVTALGASLCSSGQVASVICSGNQSQTLSPCNSSSSDPSSSIISEESGVACIGSGQLRLVDGGGRCAGRVEVYPGASWGTICDDSWDLNDAHVVCKQLSCGWAINATGSAHFGEGTGPIWLDEINCNGKESHIWQCHSHGWGRHNCRHKEDAGVICSEFMSLRLISENSRETCAGRLEVFYNGAWGSVGRNSMSPATVGVVCRQLGCADRGDISPASSDKTVSRHMWVDNVQCPKGPDTLWQCPSSPWKKRLASPSEETWITCANKIRLQEGNTNCSGRVEIWYGGSWGTVCDDSWDLEDAQVVCRQLGCGSALEAGKEPAFGQGTGPIWLNEVKCKGNEPSLWDCPARSWGHSDCGHKEDAAVTCSEIAKSRESLHATGRSSFVALAIFGVILLACLIAFLIWTQKRRQRQRLSVFSGGENSVHQIQYREMNSCLKADETDMLNPSGDHSEVQ

SEQ ID NO: 20 - DNA, 3400 bp, synthetic construct
  source 1..3400: mol_type = other DNA
atggtgctacttgaagactctggatctgcagactttagaagatgttctgcccatttaagttccttcacttttgctgtagtcgctgttctcagtgcctgcttggtcactagttctcttggaggaaaagacaaggagctgaggctaacgggtggtgaaaacaagtgctctggaagagtggaggtgaaagtgcaggaggagtggggaactgtgtgtaataatggctgggacatggatgtggtctctgttgtttgtaggcagctgggatgtccaactgctatcaaagccactggatgggctaattttagtgcaggttctggacgcatttggatggatcatgtttcttgtcgagggaatgagtcagctctctgggactgcaaacatgatggatggggaaagcataactgtactcaccaacaggatgctggagtaacctgctcagatggatctgatttagagatgaggctggtgaatggaggaaaccggtgcttaggaagaatagaagtcaaatttcaagagcggtggggaacagtgtgtgatgataacttcaacataaatcatgcttctgtggtttgtaaacaacttgaatgtggaagtgctgtcagtttctctggttcagctaattttggagaaggttctggaccaatctggtttgatgatcttgtatgcaatggaaatgagtcagctctctggaactgcaaacatgaaggatggggaaagcacaattgcgatcatgctgaggatgctggagtgatttgcttaaatggagcagacctgaaactgagagtggtagatggactcactgaatgttcaggaagattggaagtgaaattccaaggagaatggggaacaatctgtgatgatggctgggatagtgatgatgccgctgtggcatgtaagcaactgggatgtccaactgctgtcactgccattggtcgagttaacgccagtgagggaactggacacatttggcttgacagtgtttcttgccatggacacgagtctgctctctggcagtgtagacaccatgaatggggaaagcattattgcaatcataatgaagatgctggtgtgacatgttctgatggatcagatctggaactgagacttaaaggtggaggcagccactgtgctgggacagtggaggtggaaattcagaaactggtaggaaaagtgtgtgatagaagctggggactgaaagaagctgatgtggtttgcaggcagctgggatgtggatctgcactcaaaacatcatatcaagtttattccaaaaccaaggcaacaaacacatggctgtttgtaagcagctgtaatggaaatgaaacttctctttgggactgcaagaattggcagtggggtggacttagttgtgatcactatgacgaagccaaaattacctgctcagcccacaggaaacccaggctggttggaggggacattccctgctctggtcgtgttgaagtacaacatggagacacgtggggcaccgtctgtgattctgacttctctctggaggcggccagcgtgctgtgcagggaactacagtgcggcactgtggtttccctcctggggggagctcactttggagaaggaagtggacagatctgggctgaagaattccagtgtgaggggcacgagtcccacctttcactctgcccagtagcaccccgccctgacgggacatgtagccacagcagggacgtcggcgtagtctgctcaagatacacacaaatccgcttggtgaatggcaagaccccatgtgaaggaagagtggagctcaacattcttgggtcctgggggtccctctgcaactctcactgggacatggaagatgcccatgttttatgccagcagcttaaatgtggagttgccctttctatcccgggaggagcaccttttgggaaaggaagtgagcaggtctggaggcacatgtttcactgcactgggactgagaagcacatgggagattgttccgtcactgctctgggcgcatcactctgttcttcagggcaagtggcctctgtaatctgctcagggaaccagagtcagacactatccccgtgcaattcatcatcctcggacccatcaagctctattatttcagaagaaagtggtgttgcctgcatagggagtggtcaacttcgcctggtcgatggaggtggtcgttgtgctgggagagtagaggtctatcctggggcatcctggggcaccatctgtgatgacagctgggacctgaatgatgcccatgtggtgtgcaaacagctgagctgtggatgggccattaatgccactggttctgctcattttggggaaggaacagggcccatttggctggatgagataaactgtaatggaaaagaatctcatatttggcaatgccactcacatggttgggggcggcacaattgcaggcataaggaggatgcaggagtcatctgctcagagttcatgtctctgagactgatcagtgaaaacagcagagagacctgtgcagggcgcctggaagttttttacaacggagcttggggcagcgttggcaggaatagcatgtctccagccacagtgggggtggtatgcaggcagctgggctgtgcagacagaggggacatcagccctgcatcttcagacaagacagtgtccaggcacatgtgggtggacaatgttcagtgtcctaaaggacctgacacactatggcagtgcccctcatctccatggaagaagagactggccagcccctcagaggagacatggatcacatgtgccaacaaaataagacttcaagaaggaaacactaattgttctggacgtgtggagatctggtacggaggttcctggggcactgtgtgtgacgactcctgggaccttgaagatgctcaggtggtgtgccgacagctgggctgtggctcagctttggaggcaggaaaagagcccgcatttggccaggggactgggcccatatggctcaatgaagtgaagtgcaaggggaatgaaccctccttgtgggattgtcctgccagatcctggggccacagtgactgtggacacaaggaggatgctgctgtgacgtgctcagaaattgcaaagagccgagaatccctacatgccacaggtcgctcatcttttgttgcacttgcaatctttggggtcattctgttggcctgtctcatcgcattcctcatttggactcagaagcgaagacagaggcagcggctctcagttttctcaggaggagagaattctgtccatcaaattcaataccgggagatgaattcttgcctgaaagcagatgaaacggatatgctaaatccctcaggagaccactctgaagtacaatgaaaaggaaaatgggaattataacctggtgagttcagcctttaagataccttgatgaagacctggacta